(19) United States
(12) Patent Application Publication
(10) Pub. No.: US 2021/0259873 A1
(43) Pub. Date: Aug. 26, 2021

(54) Abdominal and lumbar supporting device

(71) Applicant: Oksana Lyzhyn, New York, NY (US)

(72) Inventor: Oksana Lyzhyn, New York, NY (US)

(21) Appl. No.: 16/798,252

(22) Filed: Feb. 21, 2020

(51) Int. Cl. A61F 5/02; A41C 1/02; A41C 1/10; A41F 1/04

(52) U.S. Cl. CPC A61F 5/028; A41F 1/04; A41C 1/10; A41C 1/02

(57) ABSTRACT

The present invention relates to an abdominal and lumbar supporting device in the form of corset which is worn during different stages of pregnancy and different stages of postpartum and worn during medical condition which extend the size of the lower abdomen. The device allows a user to continuously wear something that is both comfortable and discrete. The multi-layered supporting device comprises of a flat or sack like front panel which is connected by elastics to two back panels, connected to each other by adjustable laces. The front panel is also overlaid by elastic bands placed in a flat or spherical manner with their wings connected to left and right back panels, which provides additional support for the abdomen. It is possible to connect and disconnect the wings as the user desires. The device may further include a removable boning for additional abdomen and lumbar support.

### SUMMARY OF THE INVENTION

The main purpose of the invention is to provide a device that supports the abdomen and lower back during different stages of pregnancy and different stages of postpartum and during a medical condition which enlarges the size of the lower abdomen. The multi-layered supporting device comprises of a flat, or sack like, front panel which is connected by elastics to two back panels connected to each other by adjustable laces The front and back panels are comprised of 100% cotton material. The front 100% cotton panel of the device with overlapping elastic bands includes pockets for activity monitoring sensors. Additionally, the front and back cotton panels include pockets for removable plastic boning which offers additional support.

More information regarding the specific components of the invention as well as their functions will be disclosed in the descriptions that follow. These descriptions will also reference the drawings attached in order to provide for a fuller image and better understanding of the invention and its suggested uses and functions.

Let it be noted that the device being described isn't limited to the uses it is prescribed to in the description. Furthermore, the structure of the device can be slightly altered while still utilizing the same basic principles as detailed in the description and other sections. In the description, many terms are used that may put limits on the design of the product, however, this should not be regarded as so and the terms are merely a means of providing a fuller description and are not intended to be interpreted as limitations to the device models.

### DETAILED DESCRIPTION OF THE INVENTION

In the drawings, the device is illustrated in several parts. The device is composed of the front and back panels with various functional parts connecting them to ensure maximum support and adjustability to the user's body.

The front panel of the device comprises of a sack like base 1 is in a sack like shape, made out of cotton. The center of 1 has a pocket 6 which can hold a plastic bone to ensure a more sturdy support. The boning structure 6 of the device comprises of several such pockets where flexible plastic bones can be inserted. The bones are sturdy yet flexible, ensuring the best possible support and fit. The left and right sides of base 1 have pockets 10 which can hold monitoring sensors to monitor user's activities or activities of the user's fetus.

The front panel also has two straps overlapping the base. Each strap comprises of two overlapping pieces of elastic. Elastic bands 4.1, 4.2, 4.3, 4.4 are all the same size and are made of the same material. The elastic bands 4.1, 4.2, 4.3, 4.4 are made out of a flexible stretchy elastic material that both provides strong support but also bends and adjusts to fit the shape of the user. Elastic bands 4.2 and 4.4 are located slightly lower than bands 4.1 and 4.3 accordingly. Bands 4.1 and 4.3 also overlap with bands 4.2 and 4.4. In another configuration, which depends on the size of the abdomen expansion, each strap comprises of three overlapping pieces adding 4.5 and 4.6 the same size elastic band to two straps or only one wide piece of elastic band as showed in FIGS. 4 and 6. The bands are all secured in their positions in the middle of the front panel, sewn on at the same spot as the bone in the middle of the front panel. The two pairs of bands are also sewn together at the ends of the elastic straps 5. The end of the elastic straps 5 is a cotton segment that keeps the elastic bands together but isn't very stretchy. Button holes 8.2, or hooks in the case of a hook-and-eye closure, are also sewn on to the ends of the straps 5. On the interior side of the front panel there are hooks of the hook-and-eye closure 7. The hooks are made out of a hard and sturdy material such as metal.

The hooks connect to the holes located on the exterior of the strap that connects to the elastic straps 3. The piece of the device with the holes on it 7 goes underneath the front panel to find the hooks that will attach to the hooks thus connecting the front and back panels. In an alterior configuration, instead of the hook-and-eye closure, a Velcro Hook-and-Loop fastener or regular button with accompanying button holes could be used.

The back panel is comprised of two parts 2 which are made out of cotton. Each part 2 has a bone pocket and bone 6 sewn onto the side that is closer to the opposite part. The bones 6 are once again made out of a flexible plastic material that provides additional support to the device. The two parts of the back panel 2 are connected by laces 9 which can be adjusted to best fit the user. The laces 9 can be pulled tighter or loosened depending on what the user feels fits their needs best. The back panel is attached to the front panel by two strips of elastic material 3. The strips 3 are sewn on one on each side of the back panel and are made of a flexible, stretchy material to make sure the device is capable of adjusting to fit the user.

On each elastic strip 3 two buttons (or eyes) 8.1 are sewn on. The buttons (or eyes) 8.1 align to connect and fit into the button holes (or hooks) 8.2 on the ends of the straps 5. The buttons 8.1 are sewn on to the side of the strips 3 that is closer to the back panel. The button holes (or hooks) 8.2 attach to the buttons (or eyes) 8.1 and the stretchy elastic material of 4.1, 4.2, 4.3, 4.4, 4.5, 4.6 stretches accordingly to make sure it fits around the shape of the abdomen and lumbar to provide maximum support to the user. The elastic strips 3 help make sure that elastic straps 4.1, 4.2, 4.3, 4.4, 4.5, 4.6 aren't the only place receiving the weight and so that the weight is balanced throughout the user's abdomen and doesn't focus on one specific part. The components of the device all work together to adjust to the shape of the user as time progresses and provide the most support and comfort possible while also being small and discreet.

### REFERENCES CITED

Potter, M. D; U.S. Pat. No. 1,356,402; 1971
Lunney, H. J.; U.S. Pat. No. 2,166,312; 1939
Buchanan, V.; U.S. Pat. No. 2,281,533; 1942
Scott, E. M.; U.S. Pat. No. 2,285,619; 1942
Hawes, G. E.; U.S. Pat. No. 2,302,216; 1942
Amyot, L. J. A.; U.S. Pat. No. 2,305,509; 1942
Mayer, E.; U.S. Pat. No. 2,326,476; 1943
Kispert, J. J.; U.S. Pat. No. 2,334,712; 1943
Nakayama; U.S. Pat. No. 3,623,488; 1971
Moore; U.S. Pat. No. 4,005,715; 1976
Horiuchi; U.S. Pat. No. 4,643,174; 1984
Burke; U.S. Pat. No. 4,952,192; 1990
Grim; U.S. Pat. No. 4,993,409; 1991
Marcus; U.S. Pat. No. 5,060,639; 1991
Guilani et al.; U.S. Pat. No. 6,620,026; 2003
Jaccard; U.S. Pat. No. 7,765,619; 2010
Hendrickson et al.; U.S. Pat. No. 7,900,276; 2011
Christensen; U.S. Pat. No. 7,988,527; 2011
Oz; U.S. Pat. No. 8,396,229; 2013
McCrane; U.S. Pat. No. 9,975,025; 2018
Williams et al.; U.S. Pat. No. 9,987,159; 2018
Putrello, Jr.; U.S. Pat. No. 9,999,291; 2018
Seering et al.; U.S. Pat. No. D313,290; 1990
McDonald et al.; U.S. Pat. No. D374,547; 1996
Goodman; U.S. Pat. No. D658,350; 2012
Safko et al.; U.S. Pat. No. D749,230; 2016
Gomes; U.S. Pat. No. D769,578; 2016
Koppuzhayil et al.; U.S. Pat. No. D782,155; 2017

### BRIEF DESCRIPTION OF THE DRAWINGS

FIG. 1 is a top view of the exterior side of a garment designed in accordance with the device description as well as the top view of the interior of the front panel (detailed in later descriptions) of a garment designed in accordance with the device description FIG. 2 is a front view of a garment designed in accordance with the device description as it would appear on a potential user

FIG. 3 is a back view of a garment designed in accordance with the device description as it would appear on a potential user

FIG. 4 is a right side view of a garment designed in accordance with the device description as it would appear on a potential user displaying two of the potential support straps on each of the two straps of the front panel

FIG. 5 is a right side view of a garment designed in accordance with the device description as it would appear on a potential user displaying one of the potential support straps on each of the two straps of the front panel

FIG. 6 is a right side view of a garment designed in accordance with the device description as it would appear on a potential user displaying three of the potential support straps on each of the two straps of the front panel

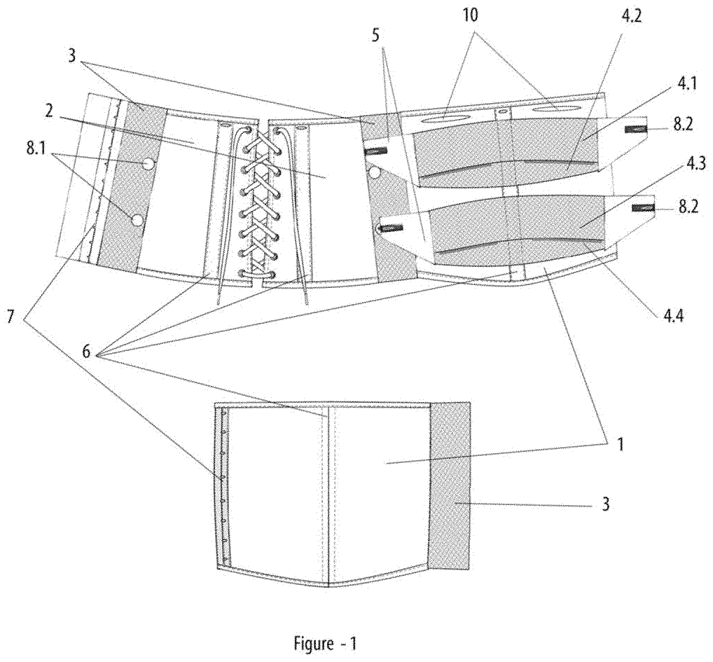

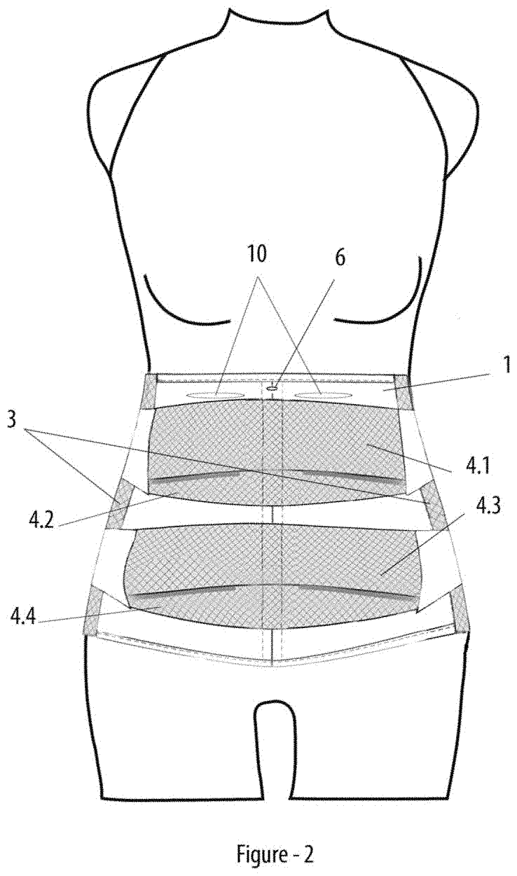

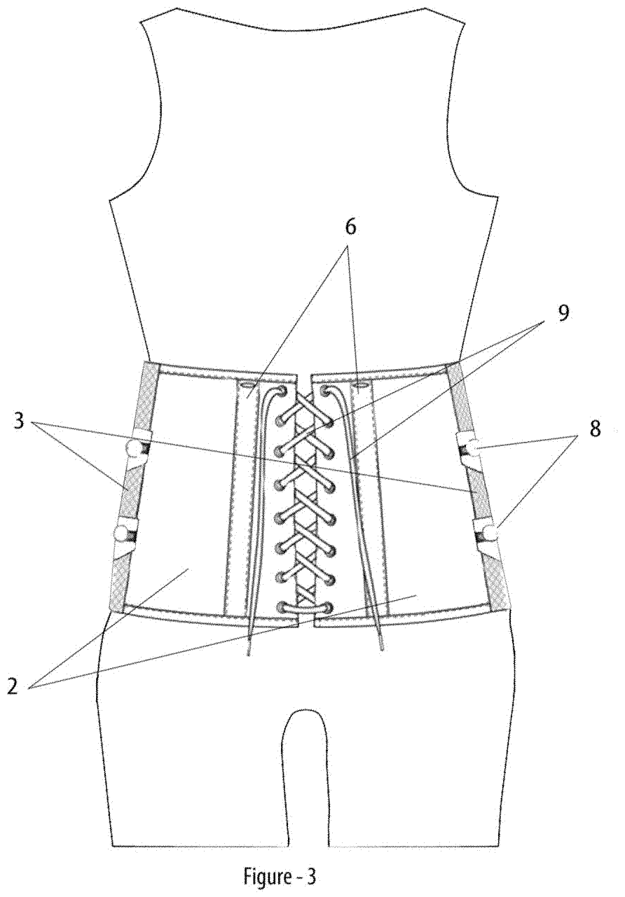

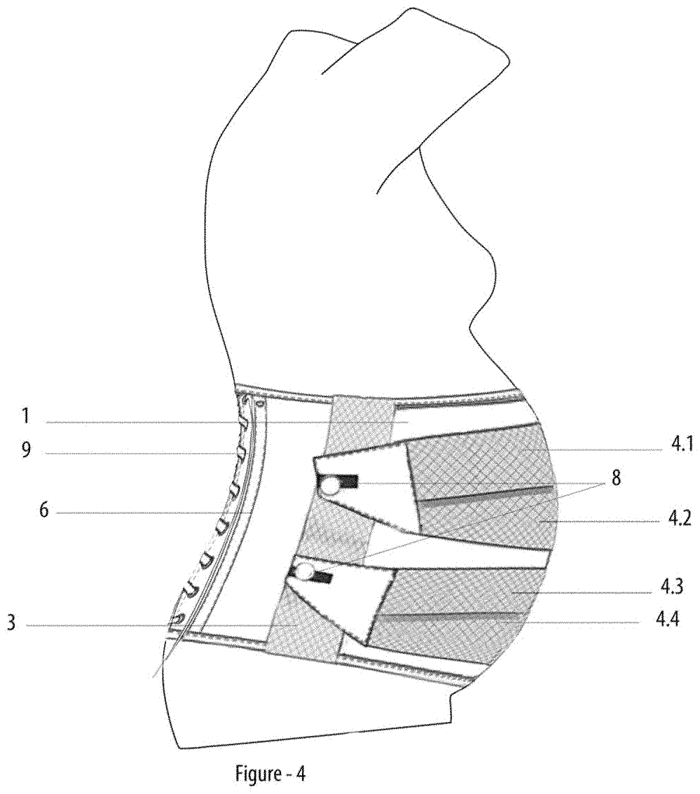

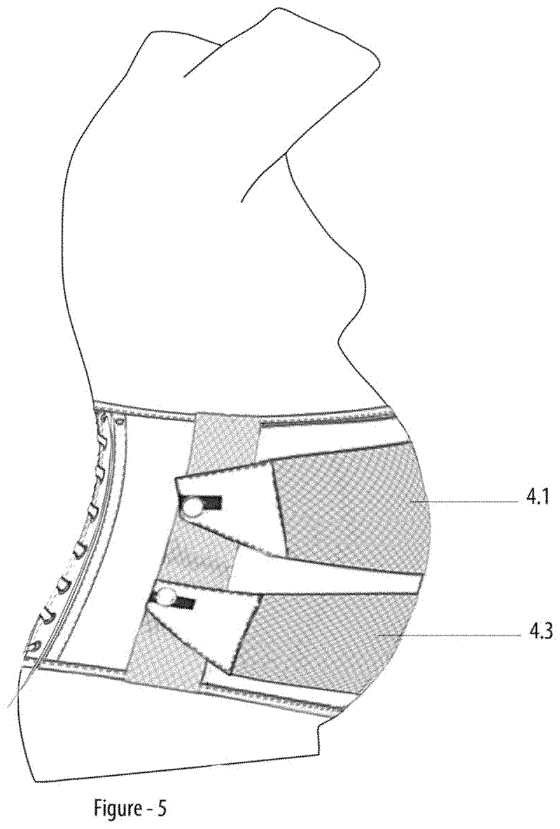

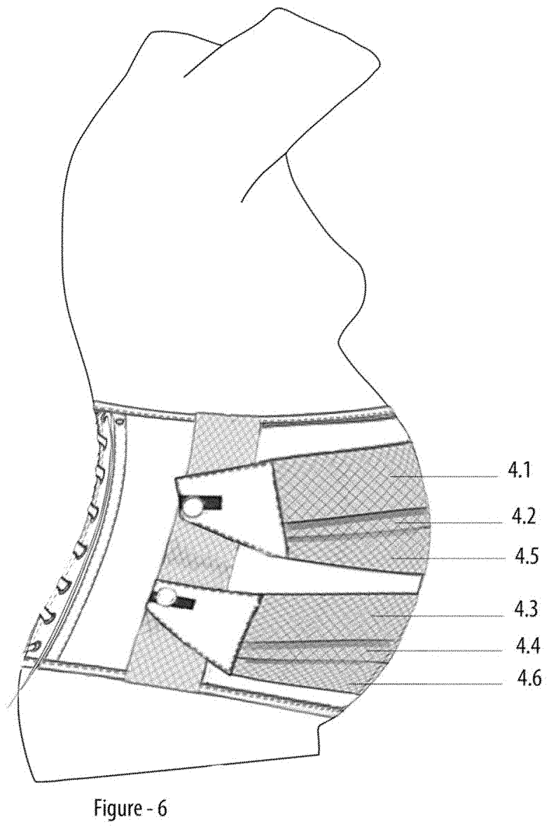

What is claimed is:

1. A device comprising:
A flat or sack like front panel connected to two back panels
Wherein device is in the form of a corset
Wherein the back panel is comprised of two parts facing each other
Wherein device assists during different stages of pregnancy and postpartum as well as in the case of a medical condition which extends the size of the lower abdomen
Wherein the device provides support for the abdomen and lumbar areas

2. the device according to claim 1 wherein the front panel is connected to the two back panels by elastic

3. the device according to claim 1 wherein the two back panels are connected to each other by adjustable laces

4. the device according to claim 1 wherein the two back panels are adjustable and can move around to best fit the user

5. the device according to claim 1 wherein the sack like front panel is made up of a main section overlapped by two smaller bands

6. the device according to claim 5 wherein the base part of the front panel is made of 100% cotton fabric

7. the device according to claim 5 wherein the straps overlapping the base of the front panel are made of elastic and are stretchable, adjusting to fit the shape of the user

8. the device according to claim 5 wherein the two straps connected to the back panels by either buttons or a hook and eye closure

9. the device according to claim 8 wherein each end of a strap is connected to one of the back panels

10. the device according to claim 7 wherein the ends of the straps are made of cotton

11. the device according to claim 5 wherein the layered structure of the front panel ensures mobility and comfort of the user as well as offering support

12. the device according to claim 5 wherein the straps are secured in the middle of the front panel

13. the device according to claim 1 wherein the front and back panels are also connected by elastic material

14. the device according to claim 13 wherein on one of the sides the elastic is attached to the front panel trough either a hook-and-eye closure, Velcro Hook-and-Loop fastener or regular button with accompanying button holes

15. the device according to claim 14 wherein at the ends of the straps and the elastic connections there are cotton straps that have the components of either the hook and eye closure, Velcro Hook-and-Loop fastener or regular buttons with accompanying button holes

16. the device according to claim 4 wherein the two back panel sections are attached to each other by laces which can be adjusted accordingly for best fit and function of device

17. the device according to claim 1 wherein the back panels have removable boning for additional comfort and support

18. the device according to claim 17 wherein the boning structure includes bones made out of sturdy yet flexible plastic material

19. the device according to claim 17 wherein there are pockets made out of cotton which the plastic bones can be inserted into

20. the device according to claim 1 wherein the device allows the user to continuously wear something that is comfortable and discrete

21. the device according to claim 1 wherein the device provides maximum support that helps users avoid discomforts during pregnancy and other medical conditions, caused by too much weight in the lower abdomen

22. the device according to claim 1 wherein there are pockets made out of cotton such that monitoring sensors to monitor user's activities or activity of the user's fetus can be inserted in
Wherein the device also prevents stretch marks and help against a number of other complications and medical conditions
